(19) United States
(12) Patent Application Publication
(10) Pub. No.: US 2016/0091500 A1
(43) Pub. Date: Mar. 31, 2016

(54) CHONDROADHERIN FRAGMENTS AS INDICATORS OF INTERVERTEBRAL DISC DEGENERATION

(71) Applicants: Lisbet HAGLUND, Montréal (CA); Peter ROUGHLEY, Beaconsfield (CA); Bashar ALKHATIB, Montréal (CA); Jean OUELLET, Westmount (CA); Patrick ÖNNERFJORD, Lund (SE)

(72) Inventors: Lisbet HAGLUND, Montréal (CA); Peter ROUGHLEY, Beaconsfield (CA); Bashar ALKHATIB, Montréal (CA); Jean OUELLET, Westmount (CA); Patrick ÖNNERFJORD, Lund (SE); Dick HEINEGARD, Lund (SE)

(21) Appl. No.: 14/891,217

(22) PCT Filed: May 13, 2014
(86) PCT No.: PCT/CA2014/050448

§ 371 Date: Nov. 13, 2015

Related U.S. Application Data
(60) Provisional application No. 61/823,326, filed on May 14, 2013.

(51) Int. Cl. G01N 33/68; C07K 16/18; C07K 14/47

(52) U.S. Cl. CPC G01N 33/6887; C07K 14/47; C07K 16/18; G01N 2800/10; G01N 2333/78

(57) ABSTRACT

The present disclosure concern fragments of the chondroadherin (CHAD) polypeptide which are generated during the proteolytic cleavage of the third leucine repeat motif of the CHAD polypeptide. Such fragments are generated and thus associated with the onset and progression of intervertebral disc degeneration and can be used to assess the risk of a subject to develop intervertebral disc degeneration or to determine if the subject is afflicted by intervertebral disc degeneration. The fragments can also be used to screen for potential therapeutic agents for preventing, treating and/or alleviating the symptoms associated with intervertebral disc degeneration.

### CROSS-REFERENCE TO RELATED APPLICATIONS AND DOCUMENTS

This application claims priority to U.S. provisional patent application 61/823,326 filed on May 14, 2013 which is incorporated herewith in its entirety. This application also comprises a sequence listing in electronic form which is incorporated herewith in its entirety.

### TECHNOLOGICAL FIELD

The present disclosure relates to biomarkers which have been measured in degenerate disc tissues but not in non-degenerate (healthy tissues). These biomarkers are proteolytic fragments of a CHAD polypeptide which can be generated by the activity of the HTRA1 protease.

### BACKGROUND

Intervertebral disc (IVD) degeneration is present in the adult with degenerative disc disease and at the apex of the spinal curves in adolescents with idiopathic scoliosis. It is characterized by structural failure and loss of IVD height due to proteolytic degradation of the extracellular matrix (ECM). Lower back pain in adult individuals is commonly associated with IVD degeneration. The societal and individual burdens for lower back pain are significant in western society, putting both physical and economic stress on the patient.

At present, little is known about the molecular mechanisms involved in IVD degeneration and how these may differ from normal turnover of the tissue. It was previously shown that chondroadherin (CHAD) is fragmented in degenerate IVD tissue from patients with scoliosis, but remains intact in macroscopically normal discs from such patients (Haglund et al. 2009).

CHAD is a leucine-rich repeat (LRR) protein mainly expressed in cartilaginous tissues, where it is located close to cells. It has the ability to bind triple helical collagen and interact with cells via the α2β1 integrin as well as via cell surface heparan sulfate proteoglycans. Interactions of CHAD with cells have been shown to lead to a variety of cellular responses with activation of intracellular signaling and changes in the cytoskeleton depending on which of the receptors are involved alone or in combination. CHAD is also expressed in other tissues that experience load, such as bone and tendon, albeit in a lower abundance.

A number of proteases have been suggested to contribute to the degenerative process in the IVD, including matrix metalloproteinases (MMPs), aggrecanases, and cathepsins. MMPs-1, -2, -3, -7, -8, -9 and -13 along with ADAMTS-4 and -5 have been shown to be upregulated in the IVD during degeneration and are responsible for breakdown of important components of the ECM, including aggrecan and collagen. Furthermore, striking expression of cathepsins K, D and L has been shown in degenerate IVD tissue. However, many of the aforementioned proteases also have significant roles in normal matrix remodeling. HTRA1 is a serine protease initially described in bacteria. Mounting evidence suggests that it plays a central role in the pathology of arthritic diseases such as osteoarthritis, and in the degradation of articular cartilage. Elevated levels of HTRA1 have also been found in degenerate IVD tissue.

It would be highly desirable to be provided with a biomarker (as well as tools to detect such biomarker) that is present in pathological conditions (such as intervertebral disc degeneration) but that is absent during normal turnover in the disc tissue. Such biomarker would preferably be able to distinguish between the normal aging process from pathological degeneration.

### BRIEF SUMMARY

One aim of the present disclosure is to provide biomarkers, as well as reagents for their detection, which are present or increased in subjects afflicted by a condition associated with an intervertebral disc degeneration when compared to subjects (e.g., age- and sex-matched) not afflicted by the condition (e.g., considered as healthy). These biomarkers can be used to determine the presence of the absence of an affliction by the condition associated with the intervertebral disc degeneration in tested subjects. These biomarkers can also be used in screening assays to assess the usefulness of an agent to prevent, treat and/or alleviate the symptoms associated to the condition associated with the intervertebral disc degeneration. As it will be shown herein, proteolytic fragments of the CHAD polypeptide, generated by cleaving the peptide bond between amino acid residues corresponding to positions 102 and 103 of SEQ ID NO: 8 (or positions 101 and 102 of SEQ ID NO: 9 or 10), are associated with the presence of intervertebral disc degeneration and are not associated with normal degeneration which can occur during aging. As it will also be shown herein is that such proteolytic fragments can be generated in vitro by incubated a intervertebral disc or cartilage In a first aspect, the present disclosure provides an isolated polypeptide comprising (and in an embodiment consisting of) a fragment of a chondroadherin (CHAD) polypeptide obtained by cleaving the CHAD polypeptide between amino acid residues corresponding to positions 102 and 103 of SEQ ID NO: 8 with a protease. In an embodiment. the CHAD polypeptide is a human CHAD polypeptide. In another embodiment, the protease is a HTRA1 protease. In still another embodiment, the isolated polypeptide is a C-terminal fragment of the CHAD polypeptide. For example, such C-terminal fragment can have as N-terminal amino acid residues, at least one of the following amino acid sequence: YLYLS (SEQ ID NO: 2), YLYLSHNDI (SEQ ID NO: 4), YLYLSHNDIR (SEQ ID NO: 5) and YLYL (SEQ ID NO: 7). In yet another example, the C-terminal fragment can have (or consist of) the amino acid sequence between amino acid residues corresponding to positions 103 and 359 of SEQ ID NO: 8. In still another embodiment, the isolated polypeptide is a N-terminal fragment of the CHAD polypeptide. For example, the N-terminal fragment can have, as C-terminal amino acid residues, at least one of the following amino acid sequence: AFRGLKQLI (SEQ ID NO: 3) and KQLI (SEQ ID NO: 6). In yet a further example, the N-terminal fragment can have (or consist of) the amino acid sequence between amino acid residues corresponding to positions 22 and 102 of SEQ ID NO: 8.

According to a second aspect, the present disclosure provides an antibody specifically recognizing the isolated polypeptide described herein. In an embodiment, the antibody can be a polyclonal antibody. The present disclosure also provides an antibody fragment specifically recognizing the isolated polypeptide described herein.

According to a third aspect, the present disclosure provides a method of characterizing an affliction by a condition associated with an intervertebral disc degeneration in a subject. Broadly, the method comprises determining the presence or the absence of the isolated polypeptide described herein in a biological sample from the subject and characterizing the subject based on such determination. The subject is characterized as being afflicted by the condition associated with the intervertebral disc degeneration if the isolated polypeptide of described herein is determined to be present in the biological sample. On the other hand, the subject is characterized as lacking the affliction by the condition associated with the intervertebral disc degeneration if the isolated polypeptide described herein is determined to be absent from the biological sample. In an embodiment, the method can further comprise determining a test level of the isolated polypeptide described herein in the biological sample from the subject and comparing the test level to a control level of the isolated polypeptide of described herein, wherein the control level is associated with a lack of affliction by the condition associated with the intervertebral disc degeneration. In such embodiment, the subject is characterized as being afflicted by the condition associated with the intervertebral disc degeneration if the test level is higher than the control level. Further, still in such embodiment, the subject is characterized as lacking the affliction by the condition associated with the intervertebral disc degeneration if the test level is the same or lower than the control level. In an embodiment, the condition is degenerative disc disease, cartilage degeneration, cartilage degeneration or scoliosis. In yet another embodiment, the biological sample is from a disc tissue and/or cerebrospinal fluid. In an embodiment, the method further comprises determining the presence or the absence of the isolated described herein or the test level of the isolated polypeptide described herein with the antibody or the antibody fragment described herein.

According to a fourth aspect, the present disclosure provides a method of characterizing an agent's ability to prevent a condition associated with an intervertebral disc degeneration. Broadly, the method comprises combining the agent with a chondroadherin (CHAD) polypeptide to provide a first mixture; adding a HTRA1 protease to the first mixture to provide a second mixture; determining the presence or the absence of the isolated polypeptide described herein in the second mixture; and characterizing the agent based on such determination. In such method, the agent is characterized as having the ability to prevent the condition associated with the intervertebral disc degeneration if the isolated polypeptide described herein is determined to be absent from the second mixture. Further, the agent is characterized as lacking the ability to prevent the condition associated with the intervertebral disc degeneration if the isolated polypeptide described herein is determined to be present in the second mixture. In an embodiment, the method further comprises determining a test level of the isolated polypeptide described herein in the second mixture; comparing the test level to a control level of the isolated polypeptide described herein, wherein the control level is associated with a lack of prevention of the condition associated with the intervertebral disc degeneration; and characterizing the agent based on such comparison. In such embodiment, the agent is characterized as having the ability to prevent the condition associated with the intervertebral disc degeneration if the test level is lower than the control level. Still in such embodiment, the agent is characterized as lacking the ability to prevent the condition associated with the intervertebral disc degeneration if the test level is equal to or higher than the control level. In an embodiment, wherein the control level is the level of the isolated polypeptide of described herein in the presence of the CHAD polypeptide and the HTRA1 protease and in the absence of the agent. In a further embodiment, the condition associated with the intervertebral disc degeneration is degenerative disc disease, cartilage degradation or scoliosis. In yet another embodiment, the method further comprises determining the presence or the absence of the isolated polypeptide described herein or the test level of the isolated polypeptide described herein 9 with the antibody or the antibody fragment described herein.

According to a fifth aspect, the present disclosure provides a method of characterizing an agent's ability to treat and/or alleviate a symptom of a condition associated with an intervertebral disc degeneration. Broadly, the method comprises combining a chondroadherin (CHAD) polypeptide with a HTRA1 polypeptide to provide a first mixture; adding the agent to the first mixture to provide a second mixture; determining the presence or the absence of the isolated polypeptide described in the second mixture; and characterizing the agent based on such determination. The agent is characterized as having the ability to treat and/or alleviate the symptom of the condition associated with the intervertebral disc degeneration if the isolated polypeptide described herein is determined to be absent from the second mixture. On the other hand, the agent is characterized as lacking the ability to treat and/or alleviate the symptom of the condition associated with the intervertebral disc degeneration if the isolated polypeptide described herein is determined to be present in the second mixture. In an embodiment, the method can further comprise determining a test level of the isolated polypeptide described herein in the second mixture; comparing the test level to a control level of the isolated polypeptide described herein, wherein the control level is associated with a lack of treatment and/or alleviation of the symptom of the condition associated with the intervertebral disc degeneration; and characterizing the agent based on such comparison. In such embodiment, the agent is characterized as having the ability to treat and/or alleviate the symptom of the condition associated with the intervertebral disc degeneration if the test level is the lower than the control level. Still in such embodiment, the agent is characterized as lacking the ability to treat and/or alleviate the symptom of the condition associated with the intervertebral disc degeneration if the test level is equal to or higher than the control level. In a further embodiment, the control level is the level of the isolated polypeptide of described herein in the first mixture. In still a further embodiment, the condition associated with the intervertebral disc degeneration is degenerative disc disease, cartilage degeneration or scoliosis. In still another embodiment, the method further comprises determining the presence or the absence of the isolated polypeptide described herein or the test level of the isolated polypeptide described herein with the antibody or the antibody fragment described herein.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides a comparison of CHAD structure in normal discs with (A) age (13, 40 or 60 years of age) and (B) disc level (T10-11, T12-L1, L2-3 or L4-5) in the spine. All protein extracts were from organ donors without degenerative disc disease (DDD) or scoliosis, and the disc level study used tissue from a 60 year old donor. Extracts were fractionated using SDS-PAGE, and immunoblotting was performed with an anti-CHAD antiserum. NP, nucleus pulposus; AF, annulus fibrosus. This data was reproduced with several samples, however for clarity only 3 representative samples are shown.

FIG. 2 provides a comparison of CHAD fragmentation in normal and degenerate disc protein extracts. Protein extracts from (A) adult discs (normal aged 60 years and degenerate aged 56 years) and (B) juvenile scoliotic discs (normal scoliotic aged 14 years and degenerate scoliotic aged 15 years) were fractionated using SDS-PAGE and immunoblotted using an anti-CHAD antiserum. NA, normal adult; DA, degenerate adult; NS, non-degenerate scoliotic; DS, degenerate scoliotic. This data was reproduced with several samples, however for clarity only 4 representative samples are shown. The ratio of fragmented to intact CHAD was evaluated in 15 non-degenerate discs aged 26 to 60 years old and 14 degenerate discs aged 15 to 70 years old (C). Protein extracts were fractionated using SDS-PAGE and band intensity was analyzed on immunoblots probed with an anti-CHAD antiserum using the ImageQuant™ TL software and a LAS4000™ image analyzer. (p=0.007)

FIG. 3 provides a comparison of CHAD fragmentation with degree of disc degeneration. Extracted proteins from the same donor (68 years of age) were fractionated by SDS-PAGE and immunoblotting was performed with an anti-CHAD antiserum. N, normal; MD, mildly degenerate; SD, severely degenerate. This data was reproduced with several samples, however for clarity only representative sample is shown.

FIG. 4 provides a (A) schematic representation of CHAD structure and location of the cleavage site characteristic of disc degeneration within the third leucine-rich repeat. C indicates the location of the 4 cysteine residues at the N-terminal and C-terminal ends of the CHAD leucine-rich repeat region. The site of the cleavage seen in vitro is marked with an arrow between isoleucine-80 and tyrosine-81. The peptide sequence on the containing isoleucine-80 is shown at SEQ ID NO: 3 whereas the peptide sequence containing the tyrosine-81 is shown at SEQ ID NO: 4. (B) MSMS spectrum of the peptide YLYLSHNDIR (SEQ ID NO: 5) with the precursor mass of 647.39 (m/z, 2+). The table below the spectrum indicates the matching MSMS fragments used for identification.

FIG. 5 provides a comparison of CHAD cleavage sites in degenerate juvenile scoliotic (15 years of age) and adult discs (normal 60 years of age, degenerate 56 years of age). Extracted proteins were fractionated by SDS-PAGE, and immunoblotting was performed using an anti-CHAD antiserum (Anti-CHAD) and an anti-neo epitope antibody (Anti-Neo, wherein the amino acid sequence of the neo epitope is shown in SEQ ID NO: 1). N, normal; DS, degenerate scoliotic; DA, degenerate adult. This data was reproduced with several samples; however for clarity only 3 representative samples are shown.

FIG. 6 shows the results of protease digests of normal disc tissue. Following digestion, extracted proteins were fractionated by SDS-PAGE and probed using an anti-CHAD antiserum. Digestions were performed with (A) MMPs 3, 7, 12 and 13; (B) ADAMTS 4 and 5; and (C) cathepsins L, B and K. −−, no enzyme control.

FIG. 7 illustrates the HTRA1 digestion of normal disc tissue. Following digestion, extracted proteins were fractionated using SDS-PAGE and probed with both an anti-CHAD antiserum digested; −−, no enzyme control; DA, degenerate adult disc extract.

FIG. 8 provides a comparison of HTRA1 protein levels in juvenile scoliotic (15 years of age) and adult discs (normal 60 years of age, degenerate 56 years of age) (A). Equivalent amounts of extracted proteins were fractionated by SDS-PAGE, and immunoblotting was performed using an anti-HTRA1 antiserum. DS, degenerate scoliotic; NA, normal adult; DA, degenerate adult. Verification of HTRA1-generated aggrecan cleavage in degenerate disc samples from 2 individuals aged 56 years and 68 years with disc tissue from a non-degenerate donor aged 60 years (B). Extracted proteins were fractionated using SDS-PAGE and immunoblotting was performed using a neoepitope antiserum directed towards the HTRA1 cleavage site in aggrecan (anti-VQTV356). NA, normal adult; DA, degenerate adult (1 and 2). This data was reproduced with multiple tissue samples; however for clarity only 3 are shown.

### DETAILED DESCRIPTION

Throughout this application, various terms are used and some of them are more precisely defined herein.

Antagonist.

This term, as used herein, refers to an agent that impedes or decreases the expression and/or activity of a HTRA1 polypeptide, especially the proteolytic cleavage of chondroadherin (CHAD) by the HTRA1 protease. An antagonist can also be a compound which decreases the biological activity (e.g., proteolytic activity) of the HTRA1 polypeptide, which downregulates the expression of a HTRA1-encoding gene, which decreases the stability of a transcript encoding the HTRA1 polypeptide or which decreases the physical association between the CHAD polypeptide and the HTRA1 protease. In the context of this disclosure, HTRA1 antagonists are considered useful for the prevention, treatment and/or alleviation of symptoms of conditions associated with an intervertrebral disc degeneration.

Antibodies.

In the context of the present disclosure, antibodies are capable of specifically recognizing the proteolytic CHAD fragments (or epitopes associated thereto). In some embodiments, the antibodies do not recognize the uncleaved (e.g., wild-type or full-length) CHAD polypeptide. Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system are known in the art. The antibodies described herein can be polyclonal or monoclonal.

In the context of the present disclosure, antibodies also include antibody derivatives such as chimeric antibodies and especially humanized antibodies. As used herein, the term “chimeric antibody” refers to an immunoglobulin that comprises both a region from two different antibodies obtained from two different animal species. As used herein, the term “humanized antibody” refers to an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human region variable region (such as a region derived from a murine antibody) capable of specifically recognizing at least one CHAD fragment and a human constant region derived from a human antibody. In another example, the humanized immunoglobulin can comprise a heavy chain and a light chain, wherein the light chain comprises a complementarity determining region derived from an antibody of non-human origin which binds to the at least one CHAD fragment and a framework region derived from a light chain of human origin, and the heavy chain comprises a complementarity determining region derived from an antibody of non-human origin which binds the at least one CHAD fragment and a framework region derived from a heavy chain of human origin.

The present disclosure also relates to fragments of the antibodies described herein. As used herein, a “fragment” of an antibody is a portion of an antibody that is capable of specifically recognizing the same epitope as the full version of the antibody. In some embodiments, antibody fragments are capable of specifically recognizing the proteolytic CHAD fragments (or epitopes associated thereto) but do not recognize the full-length or wild-type CHAD polypeptide. Antibody fragments include, but are not limited to, the antibody light chain, single chain antibodies, Fv, Fab, Fab′ and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a chimeric gene encoding the heavy chain of an F(ab′)₂ fragment can be designed to include DNA sequences encoding the CH1 domain and hinge region of the heavy chain. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e., one or more CDRs) of non-human origin and a human heavy chain framework region. The CDRs can be derived from a non-human immunoglobulin.

In some embodiments (especially for screening applications), the antibody or the antibody fragment can be coupled (i.e., physically linked) to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H. Alternatively, the antibody or the antibody fragment can be coupled to a chemotherapeutic agent; a toxin (e.g., an enzymatically active toxin of bacterial, fungal, plant, or animal origin, or fragments thereof); a radioactive isotope (i.e., a radioconjugate). Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

Biological sample. A biological sample is a sample of a subject's bodily fluid, cells or tissues. In this present disclosure, the biological sample can be derived from a disc tissue or a cartilage tissue (including articular cartilage). In some embodiments, the biological sample can be derived from blood (for example serum or plasma), cerebrospinal fluid or a fraction thereof, synovial fluid or a fraction thereof, cartilage, urine, saliva or stools. The biological sample is preferably suspected of comprising fragments of the CHAD polypeptide which are described herein. The biological sample can be used without prior modification in the various methods described herein. Optionally, the biological sample can be treated (mechanically, enzymatically, semi-purified, etc.) prior to the methods described herein.

CHAD Polypeptide.

This polypeptide, also referred to as chondroadherin, is a cartilage matrix protein thought to mediate adhesion of isolated chondrocytes. The protein contains 11 leucine-rich repeats flanked by cysteine-rich regions. In humans, it has attributed the Gene ID No. 1101. The CHAD polypeptide has been documented in humans (human precursor described in GenBank Accession Number NP_001258) and rodents (murine precursor described in GenBank Accession NP_031715.1, rat precursor described in GenBank Accession Accession: NP_062037.1). As shown herein, the cleavage of the CHAD polypeptide in its third leucin-rich repeat domain is observed in pathologic tissues (e.g., intervertebral degeneration such as degenerate disc disease and adolescent idiopathic scoliosis) and is absent from corresponding healthy tissue (which are, in an embodiment age-matched). As also shown herein, this cleavage can be simulated in vitro through the proteolytic action of the HTRA1 protease on the CHAD polypeptide. In some embodiments, the HTRA1 protease cleaves CHAD at the following cleavage site (KQLI . . . YLYL) (SEQ ID NO: 6 and SEQ ID NO: 7, respectively). The HTRA1-associated scissile bond of the CHAD polypeptide is located between amino acid residues located at positions 102 and 103 of the human CHAD polypeptide (an embodiment of which is shown in SEQ ID NO: 8) or located at positions 101 and 102 of the rodent CHAD polypeptide (embodiments of such rodent polypeptides is shown in SEQ ID NO: 9 or 10). The cleavage of CHAD by HTRA1 reveals novel epitopes in the C-terminal CHAD fragment (YLYL (SEQ ID NO: 7) which can be used to generate antibodies or associated fragments specifically recognizing CHAD's C-terminal fragment. The proteolytic CHAD fragments (N-terminal or C-terminal) can be used to distinguish between healthy subjects from those afflicted by a condition associated with a intervertebral disc degeneration as well as to screen for potential therapeutic agents for the prevention, treatment and/or alleviation of symptoms associated with intervertebral disc degeneration.

Condition Associated with an Intervertebral Disc Degeneration.

Intervertebral disc degeneration is characterized by structural failure and loss of intervertebral disc (IVD) height due to proteolytic degradation of the extracellular matrix of the disc tissue. These conditions include, but are not limited to, degenerative disc disease (adult), cartilage degradation which may occur during arthritis (such as osteoarthritis, including knee and hip arthritis for example) and idiopathic scoliosis (in adolescents). Symptoms of intervertebral disc degeneration include, but are not limited to, pain (e.g., lower back pain, neck pain, chronic pain, radiculopathy, discogenic pain, sciatica), inflammation, abonormal micro-motion instability, tingling and/or numbness in the lower limbs.

HTRA1 Polypeptide.

This polypeptide, also referred to as HtrA serine peptidase 1, is a member of the trypsin family of serine proteases. In humans, it has attributed the Gene ID No. 5654. The HTRA1 serine protease has been documented in humans (GenBank Accession Number NP_002766), in rodents (mouse version presented as GenBank Accession Number NP_062510.2, rat version presented GenBank Accession Number NP_113909.1), in cattle (cow version presented at GenBank Accession Number NP_001269011.1), in frogs (GenBank Accession Number NP_001088796.2 or NP_001072730.2), in monkeys (GenBank Accession Number NP_001245105.1 or AFE79250.1), in salmons (GenBank Accession Number NP_001135189.1 or ACI33041.1) and in marmosets (GenBank Accession Number JAB51234.1, JAB34129.1, JAB17545.1 or JAB10626.1). The HTRA1 polypeptide is provided in two forms: a 50 kDa form (also referred to as the non-processed form) and a 42 kDa form (also referred to as the processed form). As also shown herein, the non-processed form is present in both healthy and pathologic tissues, whereas the processed form is present only in pathologic tissues. As such, the 42 kDa HTRA1 form can be used to distinguish between healthy subjects from those afflicted by a condition associated with intervertebral disc degeneration.

Pharmaceutically Effective Amount or Therapeutically Effective Amount.

These expressions refer to an amount (dose) effective in mediating a therapeutic benefit to a subject (for example prevention, treatment and/or alleviation of symptoms of intervertebral disc degeneration). It is also to be understood herein that a “pharmaceutically effective amount” may be interpreted as an amount giving a desired therapeutic effect, either taken in one dose or in any dosage or route, taken alone or in combination with other therapeutic agents.

Prevention, Treatment and Alleviation of Symptoms.

These expressions refer to the ability of a method or an agent to limit the development, progression and/or symptomology of intervertebral disc degeneration. Broadly, the prevention, treatment and/or alleviation of symptoms encompass the lack of reduction of symptoms associated with intervertebral disc degeneration, such as, for example, pain.

Reaction Vessel.

The reaction vessel is an in vivo or in vitro discrete unit for characterizing a biological sample or a potential therapeutic agent. When a biological sample is being characterized, the contact between the biological sample (suspected of containing at least one CHAD fragment) and a reagent specific for the detection of at least one CHAD fragment is made under conditions suitable and for a period of time sufficient to enable the interaction between the CHAD polypeptide and the reagent. When a potential therapeutic agent is being screened, the contact between the agent, the CHAD polypeptide and the HTRA1 polypeptide must be made under conditions suitable and for a period of time sufficient to enable the agent to allow the interaction between the CHAD and HTRA1 polypeptides. Suitable in vitro environments can include, for example, a cell-free environment is combined in a reaction media comprising the appropriate reagents to enable the various measurements. Other suitable in vitro environments include cell-based assays (comprising, for example, chondrocytes, osteocytes and, in some embodiment, bone-specific matrix material) or tissue-based assays (for example using disc-derived tissues).

CHAD Fragments Associated with Intervertebral Disc Degeneration and Associated Subfragments

The present disclosure provides novel fragments, as well as associated subgfragments, of the CHAD polypeptide whose expression is associated with the onset and developement intervertebral disc degeneration. The CHAD fragments are generated from a proteolytic cleavage (and in some embodiments from the proteolytic cleavage associated with the biological activity of the HTRA1 protease) in the third leucine-rich repeat of the native (full-length) CHAD polypeptide. As shown herein, the cleavage occurs between amino acid residues corresponding to locations 102 and 103 of SEQ ID NO: 8 or between amino acid residues corresponding to locations 101 and 102 of SEQ ID NO: 9 or 10 and has been shown to generate two fragments of the CHAD polypeptide: the N-terminal fragment and the C-terminal fragment. In healthy subjects, the CHAD polypeptide is expressed in the disc tissue and is not normally detected in circulation (cerebrospinal fluid or blood). Its fragmentation and the presence of CHAD fragments in the disc tissue or circulation is associated with the onset and/or progression of intervertebral disc degeneration.

The C-terminal fragment encompasses amino acid residues located downstream (C-terminal oriented) of the proteolytic cleavage site. Such C-terminal fragment can have, as the most external N-terminal amino acid residues, the amino acid sequence: YLYLS (SEQ ID NO: 2), YLYLSHNDI (SEQ ID NO: 4), YLYLSHNDIR (SEQ ID NO: 5) or YLYL (SEQ ID NO: 7). In some additional embodiments, the amino acid sequence of the C-terminal fragments corresponding to the sequence of the amino acid residues located between positions 103 and 359 of SEQ ID NO: 8, 102 and 358 of SEQ ID NO: 9 or 102 and 358 of SEQ ID NO: 10. As shown herein, in humans, the relative size of the C-terminal fragment is about 28 kDa. In yet another embodiment, the C-terminal fragment can be located in the disc tissue and, as the degenerative condition progresses, can accumulate within the disc tissue. As such, the determination of the level of accumulation of the C-terminal fragment can be useful to determine the stage of the intervertebral disc degeneration.

The N-terminal fragment encompasses amino acid residues located upstream (N-terminal oriented) of the proteolytic cleavage site. Such N-terminal fragment can have, as the most external C-terminal amino acid residues, the amino acid sequence: AFRGLKQLI (SEQ ID NO: 3) or KQLI (SEQ ID NO: 6). In some additional embodiments, the amino acid sequence of the N-terminal fragments corresponding to the sequence of the amino acid residues located between positions 22 and 102 of SEQ ID NO: 8, 21 to 101 of SEQ ID NO: 9 or 21 to 101 of SEQ ID NO: 10. In yet another embodiment, the N-terminal fragment can be located in the disc tissue or outside the disc tissue (in the main circulation, e.g. cerebrospinal fluid or blood for example). As such, the determination of the presence or absence of the N-terminal fragment can be useful to determine the presence or the absence of an affliction by a condition associated with intervertebral disc degeneration in a subject.

The present disclosure also provides sub-fragments of the CHAD fragments described herein. These sub-fragments contain less amino acid residues than the CHAD fragments described herein and their presence is correlated with the onset and/or development of intervertebral disc degeneration. In an embodiment, the amino acid residue deletions are located at the terminus of the CHAD fragments. For example, C-terminal subfragments can have, as the most N-terminal amino acid residues the amino acid sequence: YLYLS (SEQ ID NO: 2), YLYLSHNDI (SEQ ID NO: 4), YLYLSHNDIR (SEQ ID NO: 5) or YLYL (SEQ ID NO: 7). Alternatively, the C-terminal fragment can have or consist of the amino acid sequence YLYLS (SEQ ID NO: 2), YLYLSHNDI (SEQ ID NO: 4), YLYLSHNDIR (SEQ ID NO: 5) or YLYL (SEQ ID NO: 7). A “subfragment” of the C-terminal fragment can be a polypeptide that is, for example, 4, 5, 6, 7, 8, 9 10, 15, 25, 30, 50, 75, 100, 250 or more amino acids in length. In yet another example, N-terminal subfragments can have, as the most C-terminal amino acid residues the amino acid sequence: AFRGLKQLI (SEQ ID NO: 3) or KQLI (SEQ ID NO: 6). Alternatively, the N-terminal fragment can have or consist of the amino acid sequence AFRGLKQLI (SEQ ID NO: 3) or KQLI (SEQ ID NO: 6). A “subfragment” of the N-terminal fragment can be a polypeptide that is, for example, 4, 5, 6, 7, 8, 9 10, 15, 25, 30, 50, 75 or more amino acids in length.

The present disclosure further provides antibodies capable of specifically recognizing/binding at least one of the CHAD fragment and/or subfragment and lacking the ability of specifically recognizing/binding the full-length CHAD polypeptide. As indicated above, such antibody can be a monoclonal antibody or a polyclonal antibody. Further, the antibody can either exhibit specificity towards the N-terminal fragment (including a N-terminal subfragment) or the C-terminal fragment (including a C-terminal subfragment). The antibodies can be useful for the diagnosis of a condition associated with intervertebral disc degeneration or for screening for potential therapeutic agents for the prevention, treatment and/or alleviation of symptoms of intervertebral disc degeneration.

### Predictive Applications and Associated Commercial Packages

The diagnostic and prognostic methods described herein are designed to capture the relationship between the presence (and in some embodiments, the amount) of CHAD fragments/subfragments and intervertebral disc degeneration to generate valuable information about the subject that is being characterized. Once a subject has been diagnosed by one of the predictive methods described herein, this subject can be treated according to the therapeutic regimen that is considered useful depending on its disease status. In some embodiments, the CHAD fragments/subfragments are generated can be generated due to the proteolytic activity of the HTRA1 protease.

In some embodiments, the predictive applications described herein are performed on biological samples obtained from subject who do not exhibit arthritis in large joints (e.g., hip, knee, shoulder) since such condition may bias the result obtained. Alternatively, in such subject, the predictive applications can be performed on disc tissues obtained from focal biopsies or in cerebrospinal fluid in order to limit or avoid such bias.

In the diagnostic and prognostic applications, a biological sample can be provided from the subject that is being tested. In an embodiment, the biological sample comprises a subject's tissue (for example disc tissue or cartilage tissue), cells (for example disc cells or cartilage cells), biological fluid (for example cerebrospinal fluid or synovial fluid) or polypeptides population derived therefrom. In diagnostic and prognostic applications, the biological sample can be placed in a reaction vessel. The biological sample comprises the CHAD polypeptide and is suspected of comprising the CHAD fragments or subfragments. In the assays, the reaction vessel can be any type of container that can accommodate the determination of the presence or absence of at least one CHAD fragment/subfragment and, optionally, the amount of the CHAD fragment(s)/subfragment(s) present.

Once the biological sample has been placed in the reaction vessel, it can be determined if the CHAD fragment(s)/subfragment(s) is/are present in the biological sample. In order to do so, the amino acid sequence identity of the CHAD polypeptide/fragment(s)/subfragment(s) can be determined. This determination can be made directly by sequencing the amino acid of the CHAD fragment(s)/subfragment(s) (or more specifically sequencing the amino acid identity at the C-terminal end of the N-terminal fragment/subfragment or the N-terminal of the C-terminal fragment/subfragment) present in the biological sample. In the predictive methods described herein, it is not necessary to determine the sequence identity of the complete CHAD fragment/subfragment. For example, the sequence identity of the CHAD fragment/subfragment can be determined at positions corresponding to at least one (and in some embodiments, at least two, three or four) terminal amino acid residues of the CHAD fragment/subfragment present in the biological sample. For example, the determination of the sequence identity of terminal amino acid residues corresponding to locations 98, 99, 100 and/or 101 of SEQ ID NO: 8 or 97, 98, 99 and/or 100 of SEQ ID NO: 9 or 10 may be sufficient to determine the presence of the N-terminal fragment/subfragment of the CHAD polypeptide. In another example, the determination of the sequence identity of terminal amino acid residues corresponding to locations 102, 103, 104 and/or 105 of SEQ ID NO: 8 or 101, 102, 103 and/or 104 of SEQ ID NO: 9 or 10 may be sufficient to determine the presence of the C-terminal fragment/subfragment of the CHAD polypeptide.

The determination step can be made indirectly by using reagents, In some embodiments, the determination step can rely on the addition of a qualifier specific to the sequence to be determined. The qualifier can, for example, specifically bind to a sequence or subsequence of CHAD polypeptide and/or CHAD fragment/subfragment. In those instances, the association between the qualifier and the CHAD polypeptide and/or CHAD fragment/subfragment can be used to provide the presence and, in some further embodiments, the amount of the CHAD fragment/subfragment. For example, the qualifier can be an antibody or a fragment thereof capable of specifically recognizing at least one CHAD fragments/subfragments. In some embodiments, the antibody or fragment thereof used can recognize both the CHAD polypeptide and at least one CHAD fragment/subfragment. In such embodiments, a further technique must be used to determine the presence of the CHAD fragment(s)/subfragment(s) (for example, a micro-array such as sandwich ELISA assay, a flow cytometry or a Western blot for example). The determination step may be made directly in the reaction vessel or on a sample of such reaction vessel.

Once the determination has been made, the information is extracted from the reaction vessel is can optionally be compared to corresponding control values. Such control values could be, for example, the amino acid identity, size or length of the wild-type CHAD polypeptide (amino acid residues corresponding to locations 22 to 359 of SEQ ID NO: 8 or 21 to 358 of SEQ ID NO: 9 or 10), the amino acid identity, size or length of the CHAD fragment/subfragment, the level of expression of the wild-type CHAD polypeptide in a control biological sample (from a healthy subject for example) and/or the level of expression of the CHAD fragment/subfragment in a control biological sample (from a healthy subject for example). In an embodiment, the control value is associated with a lack of intervertebral disc degeneration and, in such embodiment, the presence a CHAD fragment/subfragment is associated with a poor disease status. For example, if in the sample, it is determined that the N-terminal CHAD fragment is present or expressed at a level higher than a control value (associated for example with a level obtained from a healthy age-matched subject), the subject is characterized as being associated with a poor disease status (such as an increased prediction to develop an intervertebral disc degeneration or afflicted by an intervertebral disc degeneration). In another embodiment, the control value is associated with an intervertebral disc degeneration and, in such embodiment, the presence of at least one CHAD fragment/subfragment or in an embodiment, at a level equal to or higher than the control value, is associated with a poor disease status.

In an embodiment, the optional comparison can be made by a subject or a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to determine the presence or absence of a discrepancy between the sequence/length/size of tested CHAD fragment/subfragment with respect to sequence/length/size of the wild-type CHAD polypeptide. An output of this comparison may be transmitted to a display device. The memory, accessible by the processor, receives and stores data, such as sequence identity, number of residues, size or level of expression of the CHAD fragment/subfragment or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the optional comparison between the CHAD fragment/subfragment and the wild-type CHAD polypeptide is made, then it is possible to characterize the subject. This characterization is possible because, as shown herein, the presence of CHAD fragments/subfragments are associated with a poor disease status.

The characterization can be made by a subject or with a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to characterize the subject being tested. An output of this characterization may be transmitted to a display device. The memory, accessible by the processor, receives and stores data, such as sequence identity/length/size of the CHAD polypeptide/fragment/subfragment or any other information generated or used (such as the sequence identity, the number of residues, the size or the level of expression of the wild-type CHAD polypeptide, the CHAD fragment or the CHAD subfragment). The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The present disclosure also provides a software product embodied on a computer readable medium. This software product comprises instructions for characterizing the subject according to the methods described herein. The software product comprises a receiving module for receiving at least one of a sequence identity, a number of residues, the size or the level of expression of a CHAD fragment or a CHAD subfragment from a biological sample; a comparison module receiving input from the measuring module for determining if the sequence identity, the number of residues, the size or the level of expression is identical to the sequence of a wild-type full-length CHAD polypeptide; a characterization module receiving input from the comparison module for performing the characterization based on the comparison. In an embodiment, an application found in the computer system of the system is used in the comparison module. A measuring module extracts/receives information from the reaction vessel with respect to the sequence identity, number of residues, size or level of expression of the CHAD fragment/subfragment. The receiving module is coupled to a comparison module which receives the value(s) of the sequence identity, the number of residues, size or the level of expression of the CHAD fragment/subfragment and determines if this value is identical or different from the level of expression, the sequence and/or length of a wild-type full-length CHAD polypeptide. The comparison module can be coupled to a characterization module. In another embodiment, an application found in the computer system of the system is used in the characterization module. The comparison module is coupled to the characterization module which receives the comparison and performs the characterization based on this comparison. In a further embodiment, the receiving module, comparison module and characterization module are organized into a single discrete system. In another embodiment, each module is organized into different discrete system. In still a further embodiment, at least two modules are organized into a single discrete system.

The present disclosure also provides diagnostic and prognostic systems for performing the characterizations and methods described herein. These systems comprise a reaction vessel for placing the biological sample, a processor in a computer system, a memory accessible by the processor and an application coupled to the processor. The application or group of applications is(are) configured for receiving a level of expression, a sequence identity, a number of residue or a size of the CHAD fragment or subfragment; comparing the level of expression, the sequence identity, the number of residue or the size a wild-type CHAD polypeptide or the corresponding polynucleotide and/or characterizing the subject in function of this comparison.

The methods, softwares and systems described herein are useful for determine the predisposition of a subject to intervertebral disc degeneration. As shown herein, the presence of fragments of the wild-type CHAD polypeptide are associated with a population of subjects more susceptible of being afflicted with intervertebral disc degeneration. As such, the determination of the presence and/or amount of the CHAD fragment/subfragment can be useful in predicting the likelihood of disease in subjects being characterized. The determination of an increased susceptibility of being afflicted in such subjects can be linked to tailoring their medical regimen to include, if possible, the intake of medication to control inflammation and/or pain (for example the intake of steroids), physical therapy, exercise and/or surgery as well as to exclude, if possible, lifting heavy objects or playing sports requiring rotating the back.

The methods, softwares or systems presented herein can also be useful for diagnosing to intervertebral disc degeneration. As shown herein, the presence of fragments of the wild-type CHAD polypeptide are associated with a population of subjects being afflicted with intervertebral disc degeneration. As such, the determination of the presence and/or amount of the CHAD fragment/subfragment can be useful in predicting the presence of an affliction to a condition associated with intervertebral disc degeneration in subjects being characterized. The determination of an increased susceptibility of being afflicted in such subjects can be linked to tailoring their medical regimen to include, if possible, the intake of medication to control inflammation and/or pain (for example the intake of steroids), physical therapy, exercise and/or surgery as well as to exclude, if possible, lifting heavy objects or playing sports requiring rotating the back.

The present disclosure also provides commercial packages or kits for performing the methods described herein and assessing intervertebral disc degeneration status in a subject. The commercial package for performing the predictive applications described herewith can be used to determine the presence and/or level of at least one CHAD fragments the disc tissue (for example, from a disc tissue biopsy obtained during hernia surgery or during radio-opaque contrast injection intro the disc for diagnostic discography), in cerebrospinal fluid (for example obtained with an aspiration) and/or in a blood sample. The commercial package comprises reagents for determining the presence, the size, the sequence identity and/or level of expression of the CHAD fragment and the CHAD subfragments. In some embodiment, the reagent is an antibody or a fragment thereof (as described herein) or a combination of antibodies/fragments specific for either the wild-type CHAD polypeptide and/or the CHAD fragment/subfragment. In another embodiment, the reagent is an antibody fragment and/or a combination of antibody fragments specific for either the wild-type CHAD polypeptide or the CHAD fragment/subfragment. The commercial package can provide an ELISA assay for performing the predictive applications described herein.

Antibodies that can be used in the present methods and commercial packages are preferably those that specifically recognize the epitopes created with the cleavage of the wild-type CHAD polypeptide into CHAD fragments/subfragments. Such epitopes can include, for example, an epitope present in the motif YLYL (SEQ ID NO: 7) present in the C-terminal, in the CHAD fragment/subfragment or an epitope present in the motif KQLIA (SEQ ID NO: 6) present in the N-terminal CHAD fragment/subfragment.

### Therapeutic Applications

The present disclosure does provide that the proteloytic cleavage of the CHAD (and in some embodiment by the HTRA1 protease) is associated with the onset and the progression of intervertebral disc degeneration. Consequently, it is expected that the decreased expression or biological activity of the HTRA1 protease on the full-length CHAD polypeptide would limit or prevent some of the symptoms associated with intervertebral disc degeneration. In some embodiments, the subjects being submitted to the therapeutic agents described herein were previously determined to express at least one CHAD fragment/subfragment as described herein.

The therapeutic agents, also referred to as HTRA1 antagonists, that can be administered for this purpose include, but are not limited to, small molecules, peptides, antibodies, nucleic acids, analogs thereof, multimers thereof, fragments thereof, derivatives thereof and combinations thereof.

Administration is by any of the routes normally used for introducing a molecule into ultimate contact with blood or tissue cells. The therapeutic agents described herein can be administered in any suitable manner, preferably with the pharmaceutically acceptable carriers or excipients. The terms “pharmaceutically acceptable carrier”, “excipients” and “adjuvant” and “physiologically acceptable vehicle” and the like are to be understood as referring to an acceptable carrier or adjuvant that may be administered to a patient, together with a compound of this disclosure, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

As used herein, “pharmaceutical composition” means therapeutically effective amounts (dose) of the agent together with pharmaceutically acceptable diluents, preservatives, solubilizers, emulsifiers, adjuvants and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, and detergents (e.g., Tween 20™, Tween 80™, Pluronic F68™, bile acid salts). The pharmaceutical composition can comprise pharmaceutically acceptable solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the disclosure are particulate compositions coated with polymers (e.g., poloxamers or poloxamines).

Suitable methods of administering such nucleic acids are available and well known to those of skill in the art, and, although more than one route can be used to administer a particular composition, a particular route can often provide a more immediate and more effective reaction than another route. The preventive or therapeutic agents of the present disclosure may be administered, either orally or parenterally, systemically or locally. For example, intravenous injection such as drip infusion, intramuscular injection, intervertebral injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen.

### Screening Applications

As shown herein, CHAD fragment(s)/subfragment(s) are produced and accumulate at the onset or during the development of intervertebral disc degeneration. Without wishing to be bound by theory, it is believed that impeding or inhibiting the proteolytic cleavage of the wild-type full-length CHAD polypeptide (and in some embodiment, the cleavage mediated by the HTRA1 protease) would be useful in the prevention, treatment and/or the mitigation of symptoms associated with intervertebral disc degeneration. The present disclosure thus provides screening applications to determine the usefulness of an agent in the treatment, prevention and/or alleviation of symptoms of intervertebral disc degeneration. In the screening applications described herewith, the agent can be considered useful if it decreases the proteolytic cleavage of the CHAD polypeptide and preferably the proteolytic cleavage of the CHAD polypeptide which is mediated by the HTRA1 protease.

In order to determine if an agent would be useful for preventing intervertebral disc degeneration, an agent to be screened is contacted with a CHAD polypeptide and then a HTRA1 protease is added. In order to determine if an agent would be useful for treating and/or alleviating the symptoms of intervertebral disc degeneration, a CHAD polypeptide is contacted with a HTRA1 protease and then an agent to be screened is added. This contact may occur by placing the agent, the CHAD polypeptide and the HTRA1 protease in a reaction vessel. In the assays, the reaction vessel can be any type of container that can accommodate the measurement of a parameter of a CHAD polypeptide (such as, for example, a level of proteolytic degradation of the CHAD polypeptide or the presence of the CHAD fragment(s)/subfragment(s)).

For screening applications, a suitable in vitro environment for the screening assay described herewith can be a cell-free environment or a cultured cell. In an embodiment, the cultured cell should be able to maintain viability in culture. In such embodiment, the cultured cell(s) should express a wild-type or variant CHAD-encoding polynucleotide. The cell is preferably derived from a disc tissue (primary cell culture or cell line) and even more preferably is a nucleus pulposus cell, a cartilage cell or a chondrocyte. If a primary cell culture is used, the cell may be isolated or in a tissue-like (e.g., disc-like) structure. A further suitable environment is a non-human model, such as an animal model. If the characterization of the agent occurs in a non-human model, then the model is administered with the agent. Various dosage and modes of administration may be used to fully characterize the agent's ability to prevent, treat and/or alleviate the symptoms of intervertebral disc degeneration.

Once the contact has occured, a measurement or value of a parameter of the wild-type CHAD polypeptide is determined in the presence of the agent and the HTRA1 protease. This parameter can be, without limitation, the presence or the absence of the full-length CHAD polypeptide, the presence or the absence of the CHAD fragment(s)/subfragment(s), the biological activity of the HTRA1 protease. This assessment may be made directly in the reaction vessel (by using a probe for example) or on a sample of such reaction vessel.

The measuring step can rely on the addition of a quantifier specific to the parameter to be assessed to the reaction vessel or a sample thereof. The quantifier can specifically bind to a wild-type CHAD polypeptide, a CHAD fragment and/or a CHAD subfragment that is being assessed. In those instances, the amount of the quantifier that specifically bound (or that did not bind) to the wild-type CHAD polypeptide, a CHAD fragment and/or a CHAD subfragment is used to provide a measurement of the parameter of the wild-type CHAD polypeptide.

Various parameters of the wild-type CHAD polypeptide can be measured. For example, the parameter that is measured can be CHAD's biological activity, the polypeptide quantity, proteolytic degradation and/or stability. In another embodiment, the parameter can be the biological activity of the HTRA1 protease, specifically with respect to the proteolytic cleavage of the wild-type CHAD polypeptide. Even though a single parameter is required to enable the characterization of the agent, it is also provided that more than one parameter of the CHAD-based reagent may be measured.

The amount of the wild-type CHAD polypeptide or the CHAD fragment(s)/subfragment(s) is measured for example, through an antibody-based technique (such as a Western blot, an ELISA or flow cytometry), a micro-array, spectrometry, MRM mass spectrometry, etc. In one embodiment, this assay is performed utilizing antibodies specific to wild-type or fragments/subfragments of CHAD. Such antibodies can be directed to the surface, and unbound target or the CHAD polypeptide, fragment and/or subfragment trapped on the surface by antibody conjugation. Methods for detecting such complexes, in addition to those described above for the GST-immobilized complexes, include immunodetection of complexes using antibodies reactive with the wild-type CHAD polypeptide and/or CHAD/fragment/subfragment, as well as enzyme-linked assays which rely on detecting an enzymatic activity associated with the CHAD-based reagent or target molecule.

In some embodiments, it is also possible to evaluate the ability of screened agent to limit or inhibit the physical association of the wild-type full-length CHAD polypeptide with the HTRA1 protease. To identify such agents, a reaction mixture containing the wild-type full-length CHAD polypeptide and the HTRA1 protease is prepared, under conditions and for a time sufficient, to allow the two polypeptides to form complex. In order to test if an agent which impedes the interaction between the wild-type CHAD polypeptide and the HTRA1 polypeptide, the reaction mixture can be provided in the presence and absence of the test agent. The test agent can be initially included in the reaction mixture, or can be added at a time subsequent to the formation of the CHAD/HTRA1 complex. The formation of any complexes between the target product and the cellular or extracellular binding partner is then detected. This type of assay can be accomplished, for example, by coupling one of the components, with a label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radio-emission or by scintillation counting. Alternatively, a component can be enzymatically labeled with, for example, horseradish peroxidase, alkaline phosphatase, or luciferase, and the enzymatic label detected by determination of conversion of an appropriate substrate to product. The interaction between two molecules can also be detected, e.g., using a fluorescence assay in which at least one molecule is fluorescently labeled. One example of such an assay includes fluorescence energy transfer (FET or FRET for fluorescence resonance energy transfer). A FET binding event can be conveniently measured through standard fluorometric detection means well known in the art (e. g., using a fluorimeter). Another example of a fluorescence assay is fluorescence polarization (FP). In another embodiment, the measuring step can rely on the use of real-time Biomolecular Interaction Analysis (BIA).

In one embodiment of the screening applications, the wild-type CHAD polypeptide is anchored onto a solid phase. Examples of such solid phase include microtiter plates, test tubes, array slides, beads and micro-centrifuge tubes. In one embodiment, a CHAD chimeric polypeptide can be provided which adds a domain that allows one or both of the proteins to be bound to a matrix. Following incubation, the solid phases are washed to remove any unbound components, the matrix immobilized in the case of beads, complex determined either directly or indirectly.

Alternatively, the screening assays can be conducted in a liquid phase. In such an assay, the reaction products are separated from unreacted components, by any of a number of standard techniques, including but not limited to: differential centrifugation; chromatography (gel filtration chromatography, ion-exchange chromatography) and/or electrophoresis. Such resins and chromatographic techniques are known to one skilled in the art. Further, fluorescence energy transfer may also be conveniently utilized, as described herein, to detect binding without further purification of the complex from solution.

In addition to cell-based and in vitro assay screening systems, non-human organisms, e.g. transgenic non-human organisms or a model organism, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g. mice, rats, rabbits, guinea pigs, pigs, micro-pigs, and goats), and non-human primates (e.g. baboons, monkeys, chimpanzees) may be used in the methods described herein.

In another assay format, the specific activity or level of HTRA1-mediated proteolytic degradation of the wild-type CHAD polypeptide, normalized to a standard unit, may be assayed in a cell-free system, a cell line, a cell population or animal model that has been exposed to the agent to be tested and compared to an unexposed control cell-free system, cell line, cell population or animal model.

A transgenic cell or animal used in the methods described herein can include a transgene that encodes, e.g. a wild-type CHAD polypeptide. The transgene can encode a protein that is normally exogenous to the transgenic cell or animal, including a human protein, e.g. a human wild-type CHAD polypeptide. The transgene can be linked to a heterologous or a native promoter.

Once the measurement has been made, it is extracted from the reaction vessel and the value of the parameter of the wild-type CHAD polypeptide can optionally be compared to a control value. In an embodiment, the control value is associated with a lack of prevention, treatment and/or alleviation of symptoms of intervertebral disc degeneration. In such assay format, agents useful in the prevention, treatment and/or alleviation of symptoms of intervertebral disc degeneration are able, when compared to the control, decrease the proteolytic cleavage of the wild-type CHAD polypeptide or impede the formation of the CHAD fragment/subfragment. Alternatively or in combination, the agents identified as useful, when compared to the control, do impede or limit the biological activity of the HTRA1 protease on the wild-type CHAD polypeptide. Still in such assay format, the agents are not considered to be useful if the agent maintain or increase the proteolytic cleavage of the wild-type CHAD polypeptide or allow the formation of the CHAD fragment/subfragment. Alternatively or in combination, the agents not identified as being useful, when compared to the control, do not impede or limit the biological activity of the HTRA1 protease on the wild-type CHAD polypeptide.

In another embodiment, the control value is associated with the prevention, treatment and/or alleviation of symptoms of intervertebral disc degeneration. In such assay format, agents useful in the prevention, treatment and/or alleviation of symptoms of intervertebral disc degeneration are, when compared to the control, able to maintain or decrease the proteolytic cleavage of the CHAD polypeptide or limit the formation of the CHAD fragment/subfragment. Alternatively or in combination, the agents identified as useful, when compared to the control, impede the biological activity of the HTRA1 protease towards the wild-type CHAD polypeptide. In another embodiment, the agents are considered not to be useful if the agent increase the proteolytic cleavage of the wild-type CHAD polypeptide or allows the formation of the CHAD fragment/subfragment. Alternatively or in combination, the agents not identified as being useful, when compared to the control, do not impede or limit the biological activity of the HTRA1 protease on the wild-type CHAD polypeptide.

In the screening methods, the control value may be the parameter of the wild-type CHAD polypeptide in the absence of the agent. In this particular embodiment, the parameter of the wild-type CHAD polypeptide can be measured prior to the combination of the agent with the wild-type CHAD polypeptide or in two replicates of the same reaction vessel where one of the screening system does not comprise the agent. The control value can also be the parameter of the wild-type CHAD polypeptide in the presence of a control agent that is known not to prevent/treat/alleviate the symptoms of a proliferation-associated disease. Such control agent may be, for example, a pharmaceutically inert excipient. The control value can also be the parameter of wild-type CHAD polypeptide obtained from a reaction vessel comprising cells or tissues from a healthy subject (e.g., age- and sex-matched) that is not afflicted by intervertebral disc degeneration. The ability of the agent can be determined based on the comparison of the value of the parameter of the CHAD-based reagent with respect to the control value.

The comparison can be made by a subject or in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to determine the effect of the agent on the parameter of the CHAD polypeptide with respect to the control value. An output of this comparison may be transmitted to a display device. The memory, accessible by the processor, receives and stores data, such as measured parameters of the wild-type CHAD polypeptide or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the determination and optionally the comparison has been made, then it is possible to characterize the agent. This characterization is possible because, as shown herein, wild-type CHAD polypeptides are proteolytically cleaved and CHAD fragment(s)/subfragment(s) during the onset or the progression of intervertebral disc degeneration.

The characterization can be made by a subject or with a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to characterize the agent being screened. An output of this characterization may be transmitted to a display device. The memory, accessible by the processor, receives and stores data, such as measured parameters of the wild-type CHAD polypeptide or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The screening methods described herein can be used to determine an agent's ability to prevent, treat or alleviate the symptoms of a intervertebral disc degeneration. The premise behind this screening method is that the presence of proteolytic fragment(s) of the wild-type CHAD polypeptide is observed during the early stages of the disease (even if no symptoms are experienced by the subject) and such proteolytic fragments have been shown to accumulate within the disc tissue during the progression of the disease. As such, by assessing if the agent is capable of limiting the proteolytic cleave of the wild-type CHAD polypeptide into its corresponding CHAD fragment(s)/subfragment(s), it can be linked to its ability to prevent, treat or alleviate the symptoms of intervertebral disc degeneration.

The present disclosure also provides screening systems for performing the characterizations and methods described herein. These systems comprise a reaction vessel for placing the agent and the wild-type CHAD polypeptide, the HTRA1 protease and the agent, a processor in a computer system, a memory accessible by the processor and an application coupled to the processor. The application or group of applications is(are) configured for receiving a test value of a parameter of the CHAD polypeptide in the presence of the agent; comparing the test value to a control value and/or characterizing the agent in function of this comparison.

The present disclosure also provides a software product embodied on a computer readable medium. This software product comprises instructions for characterizing the agent according to the methods described herein. The software product comprises a receiving module for receiving a test value the wild-type CHAD polypeptide in the presence of an agent; a comparison module receiving input from the measuring module for determining if the test value is lower than, equal to or higher than a control value; a characterization module receiving input from the comparison module for performing the characterization based on the comparison.

In an embodiment, an application found in the computer system of the system is used in the comparison module. A measuring module extracts/receives information from the reaction vessel with respect to the test value of the wild-type CHAD polypeptide. The receiving module is coupled to a comparison module which receives the value(s) of the level of the CHAD polypeptide and determines if this value is lower than, equal to or higher than a control value. The comparison module can be coupled to a characterization module.

In another embodiment, an application found in the computer system of the system is used in the characterization module. The comparison module is coupled to the characterization module which receives the comparison and performs the characterization based on this comparison.

In a further embodiment, the receiving module, comparison module and characterization module are organized into a single discrete system. In another embodiment, each module is organized into different discrete system. In still a further embodiment, at least two modules are organized into a single discrete system.

The present invention will be more readily understood by referring to the following examples which are given to illustrate the invention rather than to limit its scope.

### EXAMPLE

### Characterization of CHAD Fragmentation

Materials.

The horseradish peroxidase (HRP)-conjugated anti-rabbit IgG was from Santa Cruz Biotechnology (Santa Cruz, Calif., USA). The enhanced chemiluminescence (ECL) detection system was purchased from GE Biotechnology (Baied'Urfe, Canada). The HTRA1 antibody was purchased from AbCam (Toronto, Canada). The aggrecan neo-epitope antibody for the HTRA1 cleavage site was a kind gift from Dr Zhiyong Yang in the Inflammation and remodeling research unit at Pfizer in MA. Keratanase II and chondroitinase ABC were purchased from BioLynx Inc (Brockville, Canada) and MP Biomedicals Inc (Solon, Ohio, USA), respectively. The COMPLETE® EDTA-free protease inhibitor cocktail tablets were purchased from Roche (Laval, Canada). Coomassie blue stain was purchased from Bio-Rad (Mississauga, Canada). Bovine serum albumin (BSA) was purchased from Sigma-Aldrich (Oakville, Canada). The activated keyhole limpet hemocyanin was purchased from Pierce Biotechnology (Rockford, Ill., USA). Matrix metalloproteinases and aggrecanases were all purchased from R&D systems (Minneapolis, Minn., USA). Recombinant human HTRA1 was purchased from Thermo Scientific (Waltham, Mass., USA). Recombinant cathepsins K, B and L were produced in the yeast Pichiapastoris as described (Billington et al. 2000). The polyclonal rabbit antibody recognizing CHAD was raised to the second disulphide bonded C-terminal loop. It has been tested for specificity by evaluating cross reactivity with other proteins. It only stains one band corresponding to CHAD in extracts of human articular cartilage.

Antibody Production.

A polyclonal antiserum was generated against the peptide YLYLSGGC (SEQ ID NO: 1), which was synthesized by CanPeptide (Pointe-Claire, Canada). The peptide corresponds to a 5 residue sequence from CHAD (YLYLS (SEQ ID NO: 2)) with a C-terminal linker sequence (GGC) used for coupling 4 mg of peptide to 4 mg of activated keyhole limpet hemocyanin (KLH), in accordance with the manufacturer's instructions. Immunization of rabbits with the coupled peptide for antiserum production was performed by the Comparative Medicine & Animal Resources Centre at McGill University.

Tissue Source.

Normal adult and juvenile human disc samples were obtained through the Transplant Quebec Organ Donation Program from individuals who had undergone sustained brain death. Samples were harvested within 5 hrs post-mortem. Degenerate disc samples were obtained from consenting patients undergoing discectomy and interbody fusion for discogenic axial low back pain without radiculopathy and from adolescent patients with AIS undergoing discectomy to obtain anterior release before correction of spinal deformities. The study was approved by the ethical review board at the Montreal General Hospital, Quebec, Canada.

Analysis of CHAD Fragmentation.

Disc tissue was finely diced and proteins were extracted at 4° C. under continuous agitation for 48 hrs using 15 volumes of 4 M guanidine hydrochloride, 50 mM sodium acetate, pH 5.8, 10 mM EDTA and protease inhibitors. The extracts were separated from the tissue residue by centrifugation. Aliquots of 8 μl disc extract were prepared for SDS-PAGE by precipitation using 9 volumes of 100% ethanol. Precipitated protein samples were recovered by centrifugation. To ensure that complete precipitation was achieved, the supernatant was dialyzed, concentrated and analyzed by western blotting in the same way as the precipitated protein samples. No CHAD was identified in the supernatant indicating complete precipitation. Pellets were washed once each with 75% ethanol and 95% ethanol, before being lyophilized and redissolved in 25 μl 50 mM sodium acetate, pH 6.0. This was then digested with keratanase II at 1 mU per 25 μl extract for 6 hrs. The solution was then adjusted to 100 mMTris, 100 mM sodium acetate, pH 7.3, and digested overnight with chondroitinase ABC at 50 mU per 25 μl extract. Sample buffer was added directly after digestions and the proteins were fractionated on 12% polyacrylamide gels. Proteins were transferred to nitrocellulose membranes by electroblotting (Peferoen 1988). Membranes were blocked with 1.5% (w/v) skim milk powder in 0.01 M Tris-HCl, 0.15 M NaCl, 0.1% Tween 20, pH 7.6. Antisera were diluted 1:1000 in the same buffer containing 3% BSA. Immunoblotting was performed using antibodies raised against intact CHAD or the CHAD peptide YLYLS (SEQ ID NO: 2) corresponding to a disc specific cleavage site. Bound antibodies were detected by chemiluminescence using the ECL system after incubation with a secondary antibody conjugated to horseradish peroxidase using the LAS4000 image analyzer (GE-Healthcare-Biosciences).

Ratio Analysis of Fragmented to Intact CHAD.

Band intensity was analysed on immunoblots using the ImageQuant™ TL software. A ratio was calculated for the intensity of the area representing fragmented CHAD versus the intensity of the area representing intact CHAD. Background intensity was subtracted from all samples. Quantification of CHAD fragment to intact ratios in 15 non-degenerate (average age 45 yrs, age range 26 yrs old to 60 yrs old) and 14 degenerate (average age 45.3, age range 15 yrs old to 70 yrs old) tissue donors was performed using this method. Statistical analysis was performed using unpaired T-test.

Analysis of HTRA1 Protein Levels.

Proteins were extracted and immunoblotting was performed as described in previous section. Equivalent amounts of protein were loaded in each sample well. Samples were probed using a HTRA1 antiserum diluted 1:250 in accordance with the manufacturers' instructions.

Analysis of HTRA1 Cleavage Site in Aggrecan.

Proteins were extracted and treated with keratanase II and chondroitinase ABC as described in the for the analysis of CHAD fragmentation. Equivalent amounts of protein were loaded in each sample well. Samples were probed using an aggrecan neo-epitope antiserum directed to the HTRA1 cleavage site in aggrecan (VQTV356) diluted 1:200 (Chamberland et al. 2009).

Identification of CHAD Fragmentation Site.

Proteins were separated from proteoglycans in an adult degenerate disc extract by density gradient centrifugation. Extracts were separately passed through glass wool in order to remove any tissue residue. 0.35 g CsCl was added per ml extract to obtain a density of 1.35 g/ml. Extracts were fractionated by ultracentrifugation at 100,000 gay for 72 hrs at 18° C. in a Beckman Ti50 angle rotor. Post-centrifugation, the topmost fraction, containing proteins including CHAD, was transferred into 7 M urea and further purified by ion exchange chromatography using carboxymethyl cellulose 52 (CM 52) essentially as described elsewhere (Larsson et al. 1991). Bound proteins were eluted with 0.2 M NaCl in 7 M urea, 10 mMTris, pH 7.0. CHAD-containing fractions were pooled, and fractionated by SDS-PAGE. The gel was stained with Coomassie blue, and the gel in the 28 kD region (shown to contain CHAD fragments by western blot analysis) was removed, lyophilized, reduced and alkylated and digested with trypsin. Peptides formed were identified by reversed phase liquid chromatography on-line with electrospray-iontrap mass spectrometry (LC ESI MS)(Danfelter et al. 2007).

Protease Digestion.

3 mg portions of human disc tissue (from a donor 13 years of age) were digested with the following proteases: MMP-3 (0.46 mg/ml), MMP-7, (0.43 mg/ml), MMP-12 (0.31 mg/ml), MMP-13 (0.50 mg/ml), ADAMTS4 (0.44 mg/ml), ADAMTS5 (0.43 mg/ml) and HTRA1 (0.2 mg/ml). The digestions were performed in 50 mM Tris-HCl, 200 mM NaCl, 5 mM CaCl₂, 0.01% Triton X-100. 1.8 μg of enzyme was added and the tissue was digested overnight at 37° C. For HTRA1, 1.8 μg of enzyme was added and the tissue digested for 8 hrs at 37° C., then a further 1.8 μg of enzyme was added and the reaction was continued overnight. For cathepsins, 3 mg portions of human disc tissue were digested in 2.5 mM DTT, 0.15% chondroitin sulfate A, 0.1 M sodium acetate, pH 5.5, 1 mM EDTA, overnight at 37° C. with 2.5 μg cathepsin K (0.09 mg/ml), cathepsin B (0.10 mg/ml) orcathepsin L (0.13 mg/ml).

CHAD in Non-Degenerate IVD.

CHAD fragmentation has previously been described in some IVD tissue from adolescents with Adolescent Idiopathic Scoliosis, where the level of fragmentation appeared to correlate with disc degeneration. To evaluate if CHAD fragmentation is absent in normal disc tissue throughout life, non-degenerate disc tissue from donors aged 13, 40 and 60 years of age was analyzed. Protein extracts from the nucleus pulposus (NP) and annulus fibrosus (AF) of these individuals were analyzed by SDS-PAGE and immunoblotting. CHAD was found to be present and intact in all donors regardless of age (FIG. 1A). Furthermore, intact CHAD was present at similar levels in both NP and AF in all three donors, demonstrating that CHAD is found throughout the disc.

Multiple non-degenerate discs from a 60-year-old donor were then analyzed to determine whether CHAD remained intact at all disc levels in the same individual. All discs between T10-11 and L4-5 showed the presence of only intact CHAD in both the NP and the AF regions, confirming that CHAD remains unfragmented in all non-degenerate discs irrespective of level (FIG. 1B). Thus, in both NP and AF regions of the IVD, CHAD fragmentation is absent from non-degenerate discs regardless of age or level.

CHAD Fragmentation in Degenerate IVD.

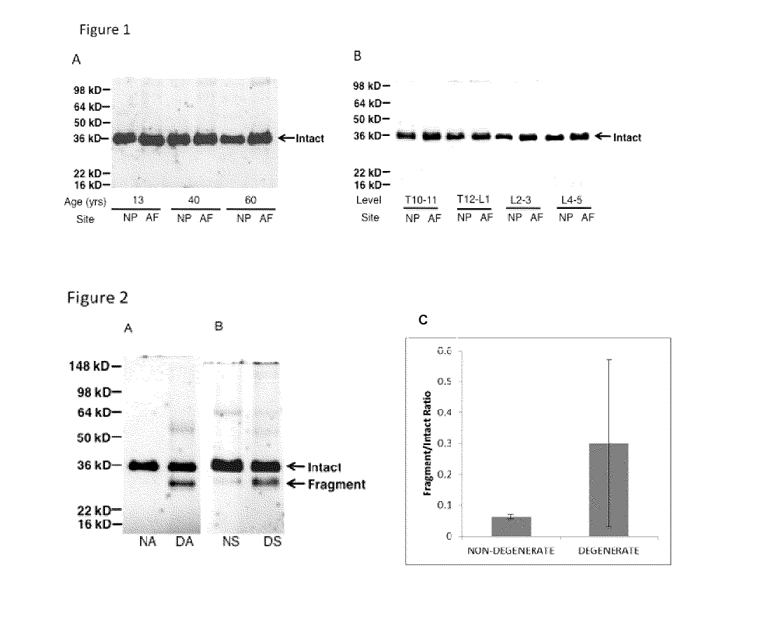

To determine if CHAD fragmentation is common to different types of disc degeneration, protein extracts from degenerate and non-degenerate discs were compared by SDS-PAGE and immunoblotting. Fragmentation of CHAD was observed in surgically excised discs from adult patients with degenerative disc disease (28 kD fragment) compared to only intact CHAD in the non-degenerate adult discs (FIG. 2A). Similarly, fragmentation of CHAD was particularly observed in the protein extracts from degenerate discs of patients with AIS, while the macroscopically normal AIS discs showed traces of the fragment probably indicating that degeneration had already started (FIG. 2B). When compared to each other, the CHAD fragment appears to possess a similar size in both the adult degenerate discs and the degenerate AIS discs. This suggests that the cleavage site responsible for CHAD fragmentation may be common in these two conditions. Furthermore, when comparing 15 non-degenerate and 14 degenerate samples, the ratio of fragmented to intact CHAD was significantly higher (p=0.007) in the degenerate samples (FIG. 2C).

Correlation of CHAD Fragmentation with Severity of Degeneration.

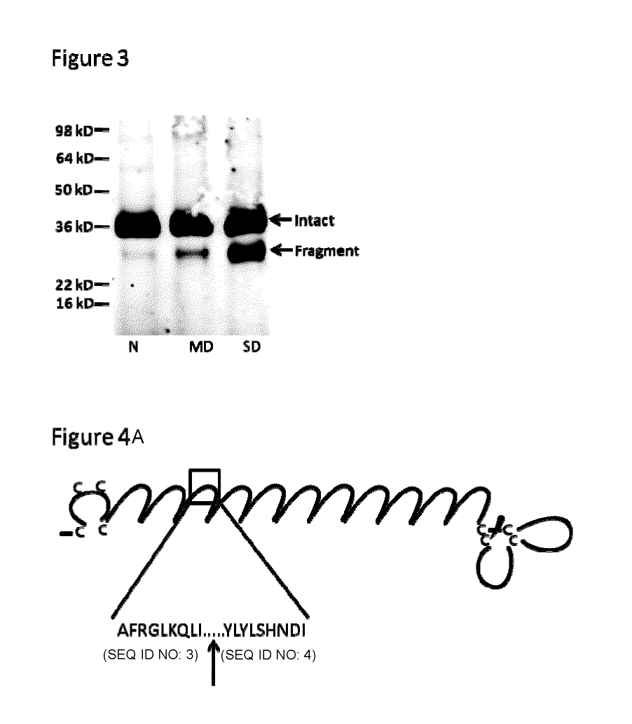

To further establish CHAD fragmentation as a marker of disc degeneration, the correlation between the level of CHAD fragmentation and severity of degeneration was studied. Punch biopsies were taken from the same degenerate disc at three different sites: macroscopically normal looking tissue, mildly degenerate tissue and severely degenerate tissue. Upon SDS-PAGE and immunoblotting analysis, CHAD was found to have little fragmentation at the macroscopically normal looking site, a small amount of fragmentation at the mildly degenerate site, and a high degree of fragmentation at the severely degenerate site (FIG. 3). Thus, with increasing degeneration, the higher becomes the abundance of CHAD fragmentation. Furthermore, irrespective of the degree of degeneration, the size of the CHAD fragment appeared constant.

Identification of the Cleavage Site of CHAD.

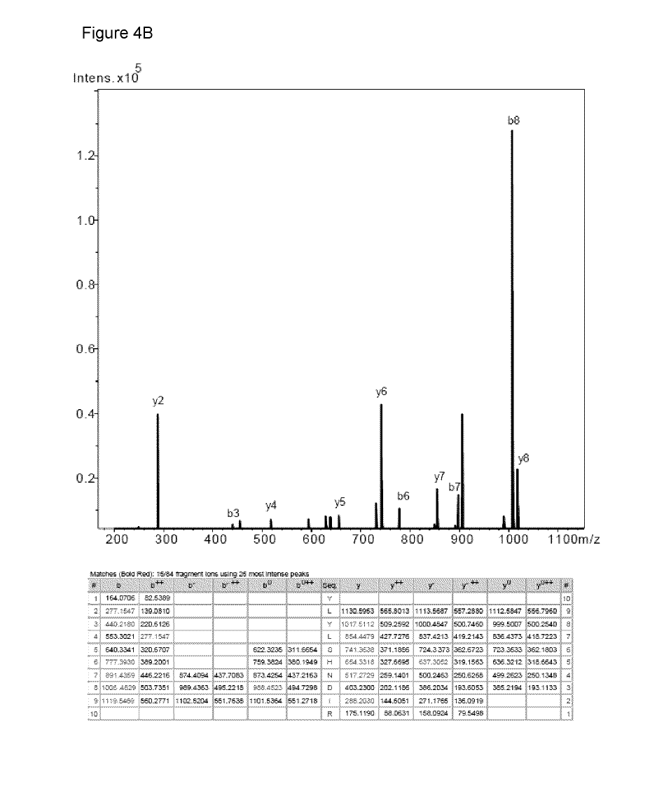

Analysis of the cleavage site at which CHAD fragmentation occurs is necessary to compare the identity of the fragments observed in adults with DDD and adolescents with AIS. For this purpose, tissue extract from a degenerate disc was fractionated by CsCl density gradient centrifugation followed by ion exchange chromatography. The CHAD-containing fractions were pooled and the proteins separated by SDS-PAGE. Gel slices were excised in the area of the gel where the fragment was present and mass spectrometric analysis was performed following trypsin digestion. A peptide “YLYLSHNDIR” (SEQ ID NO: 5) with an N-terminus not generated by trypsin digestion was identified by tandem MS resulting in a Mascot MS/MS ion score of 45 (P=0.09) where 12 b- or y-ions were matching (FIG. 4B). This sequence is found in the 3^{rd} leucine-rich repeat of CHAD (FIGS. 4A and B). Cleavage occurs between an isoleucine and a tyrosine and predicts a molecular size of 28 kD, assuming there is no additional cleavage C-terminal of this site. Furthermore, the size of the fragment generated by cleavage at this site is compatible with the size seen in the degenerated adult discs when analyzed by immunblotting.

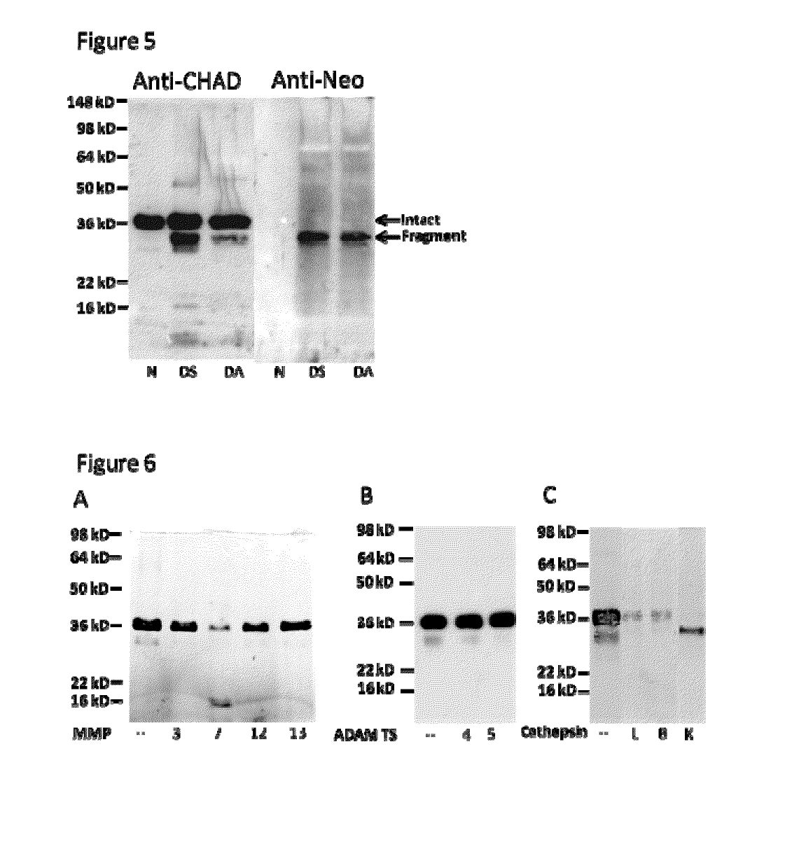

An anti-neoepitope antibody recognizing the CHAD fragment was generated by immunizing rabbits with the peptide “YLYLSGGC” (SEQ ID NO: 1) coupled to KHL. When the cleavage fragments from degenerate IVDs from both AIS and adult patients were compared by SDS-PAGE and immunoblotting analysis using the anti-neoepitope antibody, the cleavage product was found to be identical in both groups (FIG. 5). Furthermore, the single band seen in both samples when analyzed by the anti-neoepitope antibody suggests that there is no further processing at the C-terminus.

Identification of the Protease Capable of Generating the CHAD Fragment In Situ.

In order to identify the protease responsible for CHAD cleavage at the site found in situ, several proteases (MMPs, aggrecanases, cysteine and serine proteases) known to be upregulated during disc degeneration were used to digest normal disc tissue. Digestion with MMPs 3, 7, 12 and 13, showed no evidence of CHAD fragments retained in the tissue (FIG. 6A). Similarly, digestions performed with ADAMTS4 and ADAMTS5 also did not generate CHAD fragments (FIG. 6B). Digestions performed with cathepsins B and L showed extensive degradation of CHAD (FIG. 6C), though peptide fragments from these digest were too small to visualize using SDS-PAGE and immunoblotting analysis with the anti-CHAD antibody. In contrast, cathepsin K generated a CHAD fragment large enough to be retained in the gel and similar in size to that generated in situ (FIG. 6C). However, analysis performed using the anti-neoepitope antibody did not detect the fragment, demonstrating that the cleavage site was not the same as that present in situ (data not shown).

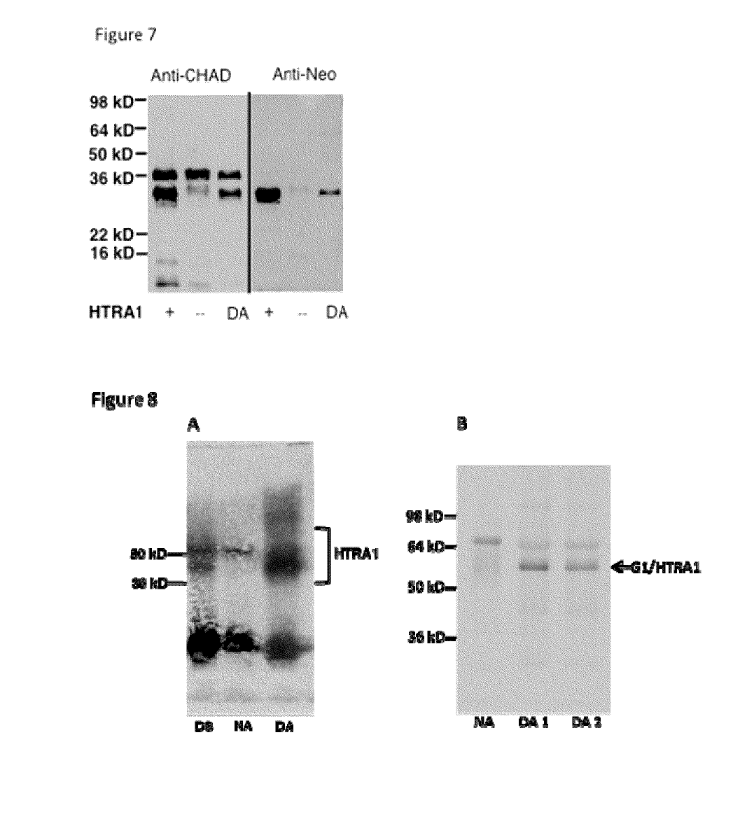

Digestions were also performed with the serine protease HTRA1. Extraction of the disc tissue and analysis via SDS-PAGE and immunoblotting demonstrated that CHAD fragments of a similar size compared to the in situ fragment were retained in the tissue (FIG. 7). Anti-neoepitope analysis of the disc tissue extract indicated that the cleavage site generated by HTRA1 was identical to that present in situ (FIG. 7).

Consequently, the ability of HTRA1 to cleave CHAD lead us to investigate the abundance of HTRA1 protein in degenerate as compared to a normal disc tissue. Elevated levels of HTRA1 protein were observed in both degenerate adult and adolescent scoliotic samples as compared to a normal disc sample (FIG. 8A). HTRA1 is represented by two bands upon analysis of the immunoblot. The higher band is common to all samples, whereas, the lower band is observed only in the degenerate samples.

HTRA-1 has also been reported to degrade aggrecan, but the presence of the resulting G1 fragment in degenerate disc tissue has not been described. Therefore, an anti-neoepitope antibody recognizing the fragment was used to investigate its presence in disc tissue. As observed for CHAD, an HTRA-1 generated aggrecan fragment was detected in degenerate disc samples, but not in normal disc samples (FIG. 8B). A band of higher molecular weight than the expected fragment was also detected in all samples. This may be due to internal epitope recognition in the aggrecanase-generated G1 domain present in this position of the gel.

In the present example, it is shown that CHAD fragmentation is a feature of disc degeneration in both the adult with DDD and in the adolescent with premature degeneration due to AIS. It was demonstrated that the site of cleavage is identical in both conditions, and shown that the protease, HTRA1, is capable of generating the CHAD cleavage site identified in IVD tissue.

Matrix homeostasis in cartilaginous tissues is maintained by a controlled turnover of the constituent macromolecules. Several proteins, cytokines, and proteases must act in concert in the disc to achieve this. A loss of balance, however, between newly synthesized macromolecules and proteases leads to the degenerative events characteristic of DDD. Disc degeneration is associated with an elevation in the expression of proteases, often as a consequence of adverse loading. This leads to proteolytic degradation of matrix components including fragmentation of proteoglycans and matrix proteins. It was found that CHAD fragmentation is present in degenerate discs, whereas in non-degenerate discs CHAD is found intact throughout life. The amount of CHAD fragmentation increases with the degree of degeneration seen in the disc. As expected some variability was found when multiple samples were compared. The degenerate group consists of samples from both organ donors with degenerate discs and surgically removed discs from patients with degenerative disc diseases, and they represents a wide range of degeneration. In discs from organ donors it is easy to separate degenerate and normal areas. This is not the case in surgical samples. It is also difficult to distinguish true non-degenerate tissue from very mildly degenerate tissue, which may explain the low signal detected in the samples designated to the non-degenerate group.

AIS presents a situation where the spine exhibits lateral curvature and a rotation of the vertebrae resulting in disc wedging and abnormal loading of the discs. This is associated with premature degeneration already in adolescent patients with the disease. Proteolysis of matrix components has been shown in donor tissue from patients with scoliosis, along with increased matrix metalloproteinase levels. Other typical signs of degeneration often seen in adult disc degeneration, such as a disorganized collagen network and cell clustering, are also found in adolescent scoliotic discs. Thus, it is not surprising that, CHAD fragmentation has also been observed in scoliotic discs showing signs of degeneration. In the present example, it was shown that CHAD is processed at the same site (KQLI . . . YLYL) (SEQ ID NO: 6 . . . SEQ ID NO: 7) in both DDD and AIS. Adverse loading is a common link between these diseases, and provides a potential common mechanism responsible for both degeneration and CHAD fragmentation. In a study by quantitative proteomics of proteins patterns in various normal cartilage tissues, including disc, fragments of CHAD were observed in only the disc samples from one individual chosen for western blot comparison to the mass spectrometry data. This finding was now followed up by western blot of the disc samples from all 5 individuals of the original study using the new neoepitope antibody. Corroborating the data presented now, there was no reactivity in any of the disc samples and the fragments previously observed clearly did not contain the epitope.

HTRA1 is a serine protease that is ubiquitously expressed in the human. Two forms of HTRA1 have been described; the larger corresponding in size with the intact protein, and the smaller suggested to represent a proteolytically processed form of HTRA1. The intact form has been reported to be present in all human discs, whereas the processed form is more abundant in degenerate discs. This has been confirmed by our study of the HTRA1 protein as a ˜50 kD protein representing the intact form is present in all samples and a ˜42 kD protein representing the processed form is more abundant in only the degenerate samples. The fact that HTRA1 is found in both degenerate and non-degenerate tissue indicates that factors other than its presence regulate its activity. As there are no known natural inhibitors to HTRA1, another mechanism for regulating enzyme activity is likely. It is possible that proteolytic processing may increase HTRA1 activity and explain why a higher level of this form is found in degenerate tissue where fragmented CHAD is also detected. An alternative explanation could be that the CHAD cleavage site is masked in the normal disc or that CHAD is not the preferred substrate for HTRA1, and that other substrates must be processed before CHAD can be degraded. Thus, CHAD would remain intact in normal disc tissue unless a threshold level of ECM proteolysis was exceeded.

In the present example, HTRA1 is the sole protease that had the capacity to cleave CHAD at the site seen in vivo in degenerate disc tissue. In contrast, MMPs 3, 7, 12 and 13, ADAMTS 4 and 5, and cathepsins K, B and L were incapable of generating the CHAD fragment found in vivo. Cathepsin K cleavage resulted in a fragment close in size to the 28 kD fragment observed in degenerate disc tissue. However, mass spectrometry analysis, of fragmented CHAD only revealed one new N-terminus in this size range corresponding to cleavage by HTRA1. Thus there is no evidence for cathepsin K activity in vivo. However, it is likely that these proteases play a role during normal disc turnover associated with development and aging, and their prior action may enhance the efficiency of CHAD cleavage by HTRA1.

Unlike CHAD, many proteins in the disc undergo degradation during normal turnover, with accumulation of fragments. Aggrecan is the most extensively studied of these molecules. It is found in cleaved and intact forms within the disc throughout life and there are numerous proteases capable of cleaving aggrecan at different sites. Metalloproteinase-mediated degradation of aggrecan has been associated with disc degeneration, but it is difficult to utilize such aggrecan fragmentation in the intervertebral disc as a specific marker, as it is not possible to distinguish degradation associated with normal tissue turnover from that associated with degeneration. It is possible that cleavage sites also exist within aggrecan which are specific to degeneration, in fact an HTRA1-generated fragment was observed present in samples also showing CHAD fragmentation using the neo-epitope antibody described by Chamberland et al. 2009 (28). However, further evaluation is needed to verify that it is not produced during normal aging and that this fragment cannot be lost due to subsequent metalloproteinase cleavage. A marker that is characterized by degradation and fragment accumulation during degeneration but that is not present during normal turnover in the disc is preferable to distinguish aging from degeneration.

The fact that CHAD is rather resistant to proteolysis by most protease implicated in normal tissue turn over provides a potential for the preservation of the HTRA1-generated cleavage fragment.

While the C-terminal fragment of CHAD may be a useful marker of degeneration within the disc, its accumulation within the disc ECM precludes it from being a useful serum marker for clinical practice. Such accumulation is probably a consequence of the retention of most of the leucine-rich repeats, which allows continued interaction of the fragment with the collagen fibrils. However, such interaction is unlikely for the corresponding N-terminal part of the protein. If this N-terminal fragment is found to be mobile and leaves the disc to enter the circulation, it could serve as cerebrospinal fluid marker or a serum marker.

It is clear that HTRA1 is not the only protease implicated in disc degeneration, and proteases from the MMP, cathepsin and ADAMTS families that are active in normal turnover could also play a role. However, as CHAD provides an important link between disc cells and the matrix in the healthy tissue, its cleavage by HTRA1 could be a pivotal step in disc degeneration. It is therefore possible that therapeutic tools aimed at inhibiting HTRA1 activity could be of value to slow down progression of disc degeneration while not influencing normal disc turnover. However, it remains to be seen whether or not there would be any adverse biological effects from systemic inhibition or whether targeted inhibition of HTRA1 within the disc is feasible.

### REFERENCES

1. Billington, C. J., Mason, P., Magny, M. C., and Mort, J. S. (2000) The slow-binding inhibition of cathepsin K by its propeptide. Biochemical and biophysical research communications 276, 924-929
2. Chamberland, A., Wang, E., Jones, A. R., Collins-Racie, L. A., LaVallie, E. R., Huang, Y., Liu, L., Morris, E. A., Flannery, C. R., and Yang, Z. (2009) Identification of a novel HtrA1-susceptible cleavage site in human aggrecan: evidence for the involvement of HtrA1 in aggrecan proteolysis in vivo. The Journal of biological chemistry 284, 27352-27359
3. Danfelter, M., Önnerfjord, P., and Heinegård, D. (2007) Fragmentation of proteins in cartilage treated with interleukin-1: specific cleavage of type IX collagen by matrix metalloproteinase 13 releases the NC4 domain. J Biol Chem 282, 36933-36941
4. Haglund, L., Ouellet, J., and Roughley, P. (2009) Variation in chondroadherin abundance and fragmentation in the human scoliotic disc. Spine (Phila Pa. 1976) 34, 1513-1518
5. Larsson, T., Sommarin, Y., Paulsson, M., Antonsson, P., Hedbom, E., Wendel, M., and Heinegård, D. (1991) Cartilage matrix proteins. A basic 36-kDa protein with a restricted distribution to cartilage and bone. J Biol Chem 266, 20428-20433
6. Peferoen, M. (1988) Blotting with plate electrodes. Methods Mol Biol 3, 395-402

1. An isolated polypeptide comprising a fragment of a chondroadherin (CHAD) polypeptide obtained by cleaving the CHAD polypeptide between amino acid residues corresponding to positions 102 and 103 of SEQ ID NO: 8 with a protease.

2. The isolated polypeptide of claim 1, wherein the CHAD polypeptide is a human CHAD polypeptide.

3. The isolated polypeptide of claim 1, wherein the protease is a HTRA1 protease.

4. The isolated polypeptide of claim 1 being a C-terminal fragment of the CHAD polypeptide.

5. The isolated polypeptide of claim 3 having, as N-terminal amino acid residues, at least one of the following amino acid sequence: YLYLS (SEQ ID NO: 2), YLYLSHNDI (SEQ ID NO: 4), YLYLSHNDIR (SEQ ID NO: 5) and YLYL (SEQ ID NO: 7).

6. The isolated polypeptide of claim 5 having the amino acid sequence between amino acid residues corresponding to positions 103 and 359 of SEQ ID NO: 8.

7. The isolated polypeptide of claim 1 being a N-terminal fragment of the CHAD polypeptide.

8. The isolated polypeptide of claim 7 having, as C-terminal amino acid residues, at least one of the following amino acid sequence: AFRGLKQLI (SEQ ID NO: 3) and KQLI (SEQ ID NO: 6).

9. The isolated polypeptide of claim 8 having the amino acid sequence between amino acid residues corresponding to positions 22 and 102 of SEQ ID NO: 8.

10. An antibody or an antibody fragment specifically recognizing the isolated polypeptide of claim 1.

11. The antibody of claim 10 being a polyclonal antibody.

12. (canceled)

13. A method of characterizing an affliction by a condition associated with an intervertebral disc degeneration in a subject, said method comprising:
determining the presence or the absence of the isolated polypeptide of claim 1 in a biological sample from the subject; and
characterizing the subject as:
being afflicted by the condition associated with the intervertebral disc degeneration if the isolated polypeptide of claim 1 is determined to be present in the biological sample; and
lacking the affliction by the condition associated with the intervertebral disc degeneration if the isolated polypeptide of claim 1 is determined to be absent from the biological sample.

14.-18. (canceled)

19. A method of characterizing an agent's ability to prevent a condition associated with an intervertebral disc degeneration, said method comprising:
combining the agent with a chondroadherin (CHAD) polypeptide to provide a first mixture;
adding a HTRA1 protease to the first mixture to provide a second mixture;
determining the presence or the absence of the isolated polypeptide of claim 1 in the second mixture; and
characterizing the agent as:
having the ability to prevent the condition associated with the intervertebral disc degeneration if the isolated polypeptide of claim 1 is determined to be absent from the second mixture; and
lacking the ability to prevent the condition associated with the intervertebral disc degeneration if the isolated polypeptide of claim 1 is determined to be present in the second mixture.

20.-23. (canceled)

24. A method of characterizing an agent's ability to treat and/or alleviate a symptom of a condition associated with an intervertebral disc degeneration, said method comprising:
combining a chondroadherin (CHAD) polypeptide with a HTRA1 polypeptide to provide a first mixture;
adding the agent to the first mixture to provide a second mixture;
determining the presence or the absence of the isolated polypeptide of claim 1 in the second mixture; and
characterizing the agent as:
having the ability to treat and/or alleviate the symptom of the condition associated with the intervertebral disc degeneration if the isolated polypeptide of claim 1 is determined to be absent from the second mixture; and
lacking the ability to treat and/or alleviate the symptom of the condition associated with the intervertebral disc degeneration if the isolated polypeptide of claim 1 is determined to be present in the second mixture.

25.-28. (canceled)


SEQUENCE LISTING (10 sequences)

SEQ ID NO: 1 - protein, 8 aa, Artificial Sequence
  Chimeric CHAD-derived peptide
YLYLSGGC

SEQ ID NO: 2 - protein, 5 aa, Homo sapiens
YLYLS

SEQ ID NO: 3 - protein, 9 aa, Homo sapiens
AFRGLKQLI

SEQ ID NO: 4 - protein, 9 aa, Homo sapiens
YLYLSHNDI

SEQ ID NO: 5 - protein, 10 aa, Homo sapiens
YLYLSHNDIR

SEQ ID NO: 6 - protein, 4 aa, Homo sapiens
KQLI

SEQ ID NO: 7 - protein, 4 aa, Homo sapiens
YLYL

SEQ ID NO: 8 - protein, 359 aa, Homo sapiens
  SIGNAL (1)..(21)
  SITE (102)..(103): Protease cleavage site
MVRPMLLLSLGLLAGLLPALAACPQNCHCHSDLQHVICDKVGLQKIPKVSEKTKLLNLQRNNFPVLAANSFRAMPNLVSLHLQHCQIREVAAGAFRGLKQLIYLYLSHNDIRVLRAGAFDDLTELTYLYLDHNKVTELPRGLLSPLVNLFILQLNNNKIRELRAGAFQGAKDLRWLYLSENALSSLQPGALDDVENLAKFHVDRNQLSSYPSAALSKLRVVEELKLSHNPLKSIPDNAFQSFGRYLETLWLDNTNLEKFSDGAFLGVTTLKHVHLENNRLNQLPSNFPFDSLETLALTNNPWKCTCQLRGLRRWLEAKASRPDATCASPAKFKGQHIRDTDAFRSCKFPTKRSKKAGRH

SEQ ID NO: 9 - protein, 358 aa, Mus musculus
  SIGNAL (1)..(20)
  SITE (101)..(102): Protease cleavage site
MARALLFSLVFLAILLPALAACPQNCHCHGDLQHVICDKVGLQKIPKVSETTKLLNLQRNNFPVLAANSFRTMPNLVSLHLQHCNIREVAAGAFRGLKQLIYLYLSHNDIRVLRAGAFDDLTELTYLYLDHNKVSELPRGLLSPLVNLFILQLNNNKIRELRAGAFQGAKDLRWLYLSENALSSLQPGSLDDVENLAKFHLDKNQLSSYPSAALSKLRVVEELKLSHNPLKSIPDNAFQSFGRYLETLWLDNTNLEKFSDAAFSGVTTLKHVHLDNNRLNQLPSSFPFDNLETLTLTNNPWKCTCQLRGLRRWLEAKASRPDATCSSPAKFKGQRIRDTDALRSCKSPTKRSKKAGRH

SEQ ID NO: 10 - protein, 358 aa, Rattus norvegicus
  SIGNAL (1)..(20)
  SITE (101)..(102): Protease cleavage site
MARVLLLSLVFLAILLPALAACPQNCHCHGDLQHVICDKVGLQKIPKVSETTKLLNLQRNNFPVLAANSFRTVPNLVSLHLQHCNIREVAAGAFRGLKQLIYLYLSHNDIRVLRAGAFDDLTELTYLYLDHNKVSELPRGLLSPLVNLFILQLNNNKIRELRAGAFQGAKDLRWLYLSENALTSLHPGSLDDVENLAKFHLDRNQLSSYPSAALSKLRVVEELKLSHNPLKSIPDNAFQSFGRYLETLWLDNTNLEKFSDAAFAGVTTLKHVHLENNRLNQLPSTFPFDNLETLTLTNNPWKCTCQLRGLRRWLEAKTSRPDATCSSPAKFKGQRIRDTDALRSCKSPTKRSKKAGRH